(19) United States
(12) Patent Application Publication
(10) Pub. No.: US 2026/0240168 A1
(43) Pub. Date: Aug. 20, 2026

(54) COMPOSITIONS AND METHODS FOR PRODUCING A FOOD CONTACT SAFE ANTIMICROBIAL COMPOSITION CONTAINING AN ESSENTIAL OIL COMPLEX

(71) Applicant: ONYX LOTUS, LLC, DOVER, DE (US)

(72) Inventor: Varun TAHLAN, DOVER, DE (US)

(21) Appl. No.: 19/161,958

(22) PCT Filed: Mar. 7, 2024
(86) PCT No.: PCT/US2024/018974

§ 371 Date: Sep. 3, 2025

Related U.S. Application Data
(60) Provisional application No. 63/450,615, filed on Mar. 7, 2023.

(51) Int. Cl. A01N 59/20; A01N 25/04; A01N 59/16; A01N 65/22; A01N 65/24; A01N 65/28; A01N 65/44; A23B 2/733; A23B 2/788

(52) U.S. Cl. CPC A01N 59/20; A01N 25/04; A01N 59/16; A01N 65/22; A01N 65/24; A01N 65/28; A01N 65/44; A23B 2/733; A23B 2/788

(57) ABSTRACT

The present disclosure provides for food contact safe compositions containing an essential oil complex, methods for preparing the food contact safe composition containing an essential oil complex, and method of disinfecting and maintaining the pathogenic sterility of an article when exposed to food contact safe compositions. These food contact safe compositions which contain a metal ion and an essential oil complex are light-stable, non-toxic, non-corrosive, and achieve a greater than 99% kill rate against a variety of pathogens for a period of time greater than 1 day on a variety of articles.

### RELATED APPLICATIONS

This application claims the benefit of U.S. Provisional Application Ser. No. 63/450,615 entitled “Compositions and Methods for a Food Contact Safe Composition Containing an Essential Oil Complex”, filed Mar. 7, 2023, which is incorporated herein by reference in its entirety.

### FIELD

Embodiments of the instant disclosure generally relate to compositions and methods for disinfecting an article using a food contact safe composition containing an essential oil complex. The compositions and methods are effective against a variety of pathogens.

### BACKGROUND

Effective control of pathogens, such as viruses, bacteria, fungus, and mold, using various commercial disinfectants, such as bleach, sprays (e.g., Lysol®), and aqueous compositions (e.g., Pine Sol®) has been performed for a number of years. Through the use of commercial disinfectants, whether sprays or wipes, an adequate number of bacteria, fungus, and mold can be effectively reduced for a short period of time. Yet, despite this immediate reduction, the bacteria, fungi, or mold can quickly repopulate once the disinfectant has dissipated or evaporated from the surface. These commercial disinfectants generally have little or no effect on viruses and in some cases are considered corrosive and toxic. These commercial disinfectants tend not to be utilized on food packaging, food products, or in extended contact with human skin due to their corrosive nature.

Therefore, there is a need for a food contact safe composition containing an essential oil complex possessing light stable, non-toxic, and non-corrosive properties that is not only effective against different pathogens (e.g., bacteria, mold, fungi, and viruses) but also provides efficacy on a variety of surfaces such as food products, cosmetics, or for disinfectant of a wound, for an extended period of time.

### BRIEF SUMMARY

Embodiments of the present disclosure relate to compositions and methods for disinfecting a variety of articles using a food contact safe composition containing an essential oil complex. The compositions and methods are effective against a variety of pathogens. In some embodiments, the present disclosure relates to a composition for killing pathogens comprising at least one metal ion, an essential oil, an emulsifying agent, a solvent and an excipient.

In one embodiment the food contact safe composition comprises about 0.001 weight percent (wt. %) to about 2 wt. % of at least one metal ion, about 1 wt. % to about 5 wt. % of an essential oil, about 1 wt. % to about 5 wt. % of an emulsifying agent and about 88 wt. % to 98 wt. % of a solvent.

In one embodiment, the present disclosure provides methods for preparing a food contact safe composition containing an essential oil complex, the methods comprising; (a) combining the emulsifying agent, the solvent, and the at least one acceptable excipient to form a mixture; (b) combining the mixture from step (a) with an essential oil; and (c) combining the mixture from step (b) with at least one metal ion.

In one embodiment, the present disclosure provides a method for disinfecting and/or maintaining the pathogenic sterility of an article comprising contacting the article with the food contact safe composition containing an essential oil complex. In one embodiment, the food contact safe composition is applied at 0.1-1000 μL/cm² to 1-10 mL/cm².

Other features and iterations of the invention are described in more detail below.

### BRIEF DESCRIPTION OF THE FIGURES

FIGS. 1A and 1B show the results of time kill studies on Salmonella enterica with food contact safe compositions at 50% and 100% concentrations at various time intervals. FIG. 1A shows results with 50% and 100% concentrations of E3B-2. FIG. 1B shows results with 50% and 100% concentrations of E3B-3.

FIGS. 2A and 2B show the results of time kill studies on Staphylococcus aureus with food contact safe compositions at 50% and 100% concentrations at various time intervals.

FIG. 2A shows results with 50% and 100% concentrations of E3B-2. FIG. 2B shows results with 50% and 100% concentrations of E3B-3.

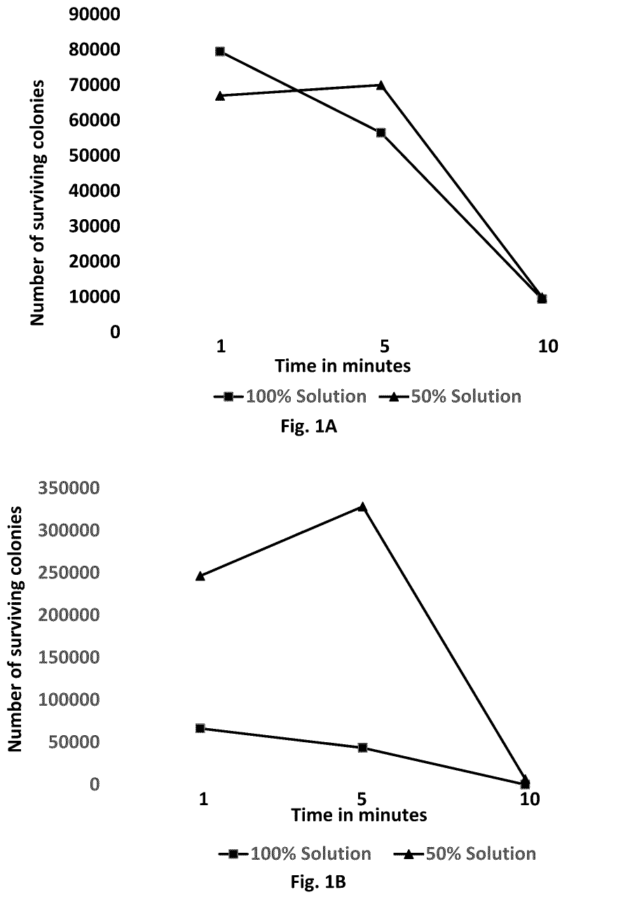

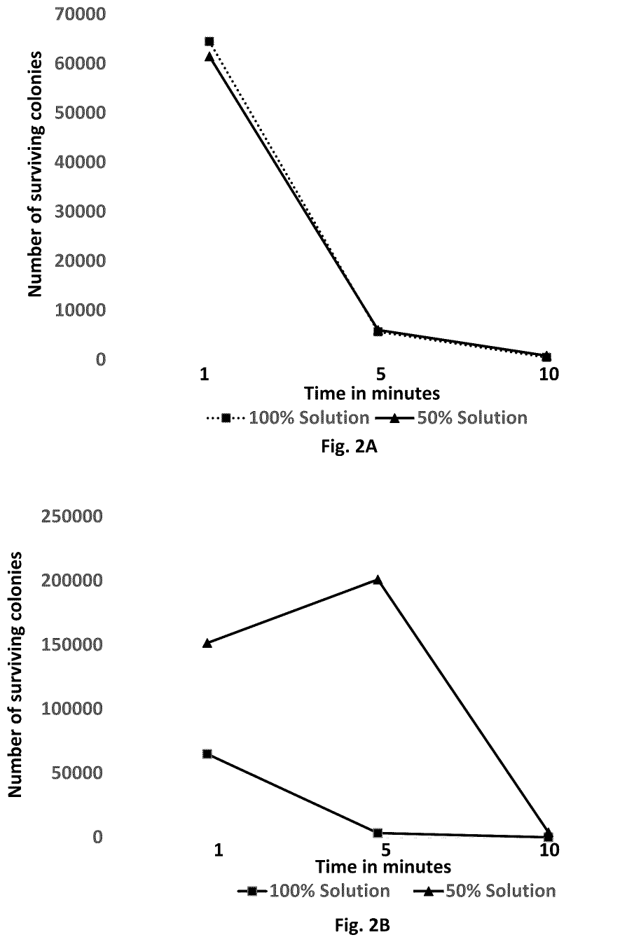

### DEFINITIONS

Unless defined otherwise, all technical and scientific terms used herein have the meaning commonly understood by a person skilled in the art to which this invention belongs.

This description will enable one skilled in the art to make and use the invention, and it describes several embodiments, adaptations, variations, alternatives, and uses of the invention. These and other embodiments, features, and advantages of the present invention will become more apparent to those skilled in the art when taken with reference to the following detailed description of the invention in conjunction with the accompanying drawings.

Reference throughout this specification to “one embodiment”, “some embodiments”, “certain embodiments”, “one or more embodiments” or “an embodiment” means that a particular feature, structure, material, or characteristic described in connection with the embodiment is included in at least one embodiment of the invention. Thus, the appearances of phrases containing the term “embodiment(s)” in various places throughout this specification are not necessarily referring to the same embodiment of the invention.

Furthermore, the particular features, structures, materials, or characteristics may be combined in any suitable manner in one or more embodiments.

In the present disclosure, “%” refers to “weight % (wt. %)” or “mass %”, unless otherwise stated.

As used herein, the phrase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phrase “consisting essentially of” limits the scope of a claim to the specified elements or method steps, plus those that do not materially affect the basis and novel characteristic(s) of the claimed subject matter.

When introducing elements of the embodiments described herein, the articles “a”, “an”, “the” and “said” are intended to mean that there are one or more of the elements.

The terms “comprises”, “comprising”, or any other variations thereof used in the disclosure, are intended to cover a non-exclusive inclusion, such that a device, apparatus, system, assembly, method that comprises a list of components or a series of steps that does not include only those components or steps but may include other components or steps not expressly listed or inherent to such apparatus, or assembly, or device. In other words, one or more elements or steps in a system or device or process proceeded by “comprises . . . a” or “comprising of” does not, without more constraints, preclude the existence of other elements or additional elements or additional steps in the system, device, or process as the case may be. Besides, the use of “comprising”, “consisting” or “including” also contemplates embodiments that “consist essentially of” or “consist of” the recited formulation and steps of preparation of the formulation.

As used herein, the term “nanoparticle”, in all of its forms, refers to a particle characterized by a particle size of less than one micron.

As used herein, the term “light-stable”, in all of its forms, refers to the disinfectant coating composition not losing efficacy or potency in the presence of light, either sunlight or manmade light.

As used herein, the phrase “contacting the article”, refers to exposing an article to a composition.

As used herein, the phrase “food contact safe”, refers to a composition that can be applied to foods without impacting the food, or impacting a mammal that consumes said food.

As used herein, the term “brine”, refers to a liquid with saturating amounts of sodium chloride or calcium chloride.

As used herein, the terms “pathogen” and “microorganism” may be used interchangeably, and refer to bacteria, fungi and viruses.

As used herein, the term “viable pathogen” refers to a pathogen or microorganism that is metabolically or physiologically active. When describing a virus, “viable pathogen” refers to a virus that is able to undergo replication.

As used herein, the term “pathogenic sterility” refers to a surface or article that has no viable pathogens present.

As used herein, the terms “antibacterial properties” or “antimicrobial properties” or “disinfectant properties” refers to the ability of a composition to remove greater than 95% of all viable pathogens.

As used herein, the term “kill” refers to the ability of a compositions to act as a biocide.

Concentrations, amounts, and other numerical data may be expressed or presented herein in a range format. It is to be understood that such a range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited. As an illustration, a numerical range of “about 2 to about 50” should be interpreted to include not only the explicitly recited values of 2 to 50, but also include all individual values and sub-ranges within the indicated range. Thus, included in this numerical range are individual values such as 2, 2.4, 3, 3.7, 4, 5.5, 10, 10.1, 14, 15, 15.98, 20, 20.13, 23, 25.06, 30, 35.1, 38.0, 40, 44, 44.6, 45, 48, and sub-ranges such as from 1-3, from 2-4, from 5-10, from 5-20, from 5-25, from 5-30, from 5-35, from 5-40, from 5-50, from 2-10, from 2-20, from 2-30, from 2-40, from 2-50, etc. This same principle applies to ranges reciting only one numerical value as a minimum or a maximum. Furthermore, such an interpretation should apply regardless of the breadth of the range, or the characteristics being described.

As used herein, the term “about” is also used to provide flexibility to a numerical range endpoint by providing that a given value may be “a little above” or “a little below” the endpoint. For example, the endpoint may be within 10%, 8%, 5%, 3%, 2%, or 1% of the listed value. Further, for the sake of convenience and brevity, a numerical range of “about 50 mg/mL to about 80 mg/mL” should also be understood to provide support for the range of “50 mg/mL to 80 mg/mL”. The endpoint may also be based on the variability an appropriate regulatory body, such as the FDA, EPA, USP, etc.

In this disclosure, the terms “including”, “containing” and/or “having” are understood to mean “comprising” and are open-ended terms.

As various changes could be made in the above-described methods without departing from the scope of the invention, it is intended that all matter contained in the above description and in the examples given below, shall be interpreted as illustrative and not in a limiting sense.

### DETAILED DESCRIPTION OF EMBODIMENTS

In the following sections, certain exemplary compositions and methods are described to detail certain embodiments of the invention. It will be obvious to one skilled in the art that practicing the certain embodiments does not require the employment of all or even some of the specific details outlined herein, but rather that concentrations, times, and other specific details can be modified through routine experimentation. In some cases, well known methods or components have not been included in the description.

The present disclosure is based partly on the discovery that metal ions in a food contact safe composition containing an essential oil complex are able to kill and reduce the number of viable pathogens, as well as the variety of pathogens, when applied to a surface or article.

The ability of the food contact safe composition to reduce the number of viable pathogens is maintained for more than 1 day and preferably more than 1 week, when applied to food products or to human skin, as a cosmetic, and/or as a pharmaceutical.

Importantly, the food contact safe composition containing an essential oil complex is light stable, economical, easily prepared, non-toxic to mammals, non-corrosive to surfaces, exhibits antimicrobial properties, antibacterial properties, antiviral properties, antifungal properties, or a combination thereof.

### I. Food Contact Safe Compositions Containing an Essential Oil Complex

The present invention relates to food contact safe compositions containing an essential oil complex with antimicrobial properties. The food contact safe compositions containing an essential oil complex include at least one metal ion, an essential oil, an emulsifying agent, an excipient and a solvent. These food contact safe compositions kill a variety of pathogens including bacteria, fungi, and viruses on the surface of food products, food storage containers, and on human skin.

The food contact safe compositions containing an essential oil complex are easily prepared, easily applied, light stable, heat stable, non-toxic to mammals, non-corrosive to surfaces, and also exhibit antimicrobial properties, antibacterial properties, antifungal properties, antiviral properties, and combinations thereof.

In one embodiment, once applied, these food contact safe compositions containing an essential oil complex kills pathogen more than 95% of pathogens on the surface of an article.

In one embodiment, the long-lasting coating kills pathogens on the surface of an article at a percent selected from the group consisting of more than 80%, 85%, 90%, 95%, 99% and 99.9999%, as well as maintaining its efficacy on a surface of an article for a period of time from 1 day to greater than 1 week or more.

In one embodiment, the food contact safe compositions maintain their efficacy in killing pathogens for a period of time selected from the group consisting of 12 hours to 6 months; one week to one month; one month to 6 months; three months to 6 months; one day to one month; 5-20 days; 1-10 days; 1-180 days; 15-180 days and 12-24 hours or combinations thereof.

The food contact safe compositions containing an essential oil complex includes at least one metal ion which is a food-safe metal ion. The at least one metal ion is considered non-toxic to mammals, when either ingested or applied topically. The at least one metal ion is derived from a water-soluble metal salt which releases a metal ion in a solvent and/or is a transition metal salt that imparts disinfectant properties.

Silver, as well as other metal ions, such as copper, zinc, gold, cobalt, zirconium, molybdenum, and palladium ions possess antimicrobial properties, antibacterial properties, and antifungal properties. Salts of these ions are considered to be active antimicrobial agents, antibacterial agents, and antifungal agents as long as a portion of the metal ion dissociates from the metal salt in the solvent (such as water, brine, stomach acid, human serum, blood, or a polar protic solvent).

These metal ions react with pathogens at low ppm (parts per million) levels in various ways, such as binding to the cell wall of the pathogen to block critical substances from passing into or out of the pathogen's cells, releasing active oxygen species which interact with the pathogen's DNA or RNA to inhibit replication of the pathogen, and/or being transported within the cell of the pathogen to block the respiratory system, thereby destroying energy production within the cell of the pathogen. By contacting the pathogen with the food contact safe composition, reactive oxygen species are released. Non-limiting examples of reactive oxygen species include but are not limited to oxygen, a superoxide anion, a peroxide anion, a hydroxyl radical, or combinations thereof. These reactive oxygen species, once in contact with a pathogen can cause damage to cells through oxidative damage. The metal ions present a positively charged surface, which interact with the negatively charged cell membrane of the pathogen causing physical damage.

The metal ion may be derived from a transition metal salt that imparts disinfectant properties. Non-limiting examples of the transition metal salt which imparts disinfectant properties are selected from the group consisting of a silver salt, a copper salt, a zinc salt, a gold salt, a cobalt salt, a zirconium salt, a molybdenum salt, a palladium salt, and combinations thereof. The anion of the at least one metal salt may have an organic or an inorganic anion. Non-limiting examples of the metal salt include but are not limited to silver nitrate, silver acetate, silver bromide, silver sulfate, silver citrate, silver oxalate, copper (II) acetate, copper (II) sulfate, copper (I) chloride (II), zinc and/or copper carbonate, zinc chloride, zinc nitrate, zinc acetate, zinc sulfate, gold acetate, gold chloride, cobalt (II) sulfate, cobalt (II) chloride, cobalt (II) nitrate, cobalt (II) carbonate, zirconium (IV) nitrate, zirconium (IV) acetate, molybdenum (II) chloride, molybdenum (V) chloride, and palladium (II) chloride.

In one embodiment, the food contact safe composition containing an essential oil complex includes a silver salt, a copper salt, a zinc salt, or a combination thereof. In one embodiment, the food contact safe composition includes a silver salt. In one embodiment, the food contact safe composition containing an essential oil complex includes a copper salt. In one embodiment, the food contact safe composition containing an essential oil complex includes a zinc salt.

In one embodiment, the food contact safe composition containing an essential oil complex includes a silver salt that is capable of releasing a silver cation, such as Ag⁺ but potentially Ag²⁺, and Ag³⁺ in addition to Ag⁺. Suitable, non-limiting examples of silver salts are silver chloride, silver bromide, silver fluoride (AgF, AgF₂, and/or Ag₂F), silver iodide, silver citrate, silver lactate, silver phosphate, silver carbonate, silver sulfate, silver trifluoroacetate, silver perchlorate, silver acetate, silver nitrate, silver oxide, silver perchlorate, and combinations thereof. In one embodiment, the silver salt is silver nitrate.

In general, the at least one metal salt is present in an amount ranging from 0.001-2 wt. % based on the total weight of the food contact safe composition containing an essential oil complex. In one embodiment, the at least one metal salt is present in an amount selected from the group consisting of 0.001-2 wt. %, 0.001 to 0.01 wt. %, 0.005-0.01 wt. %, 0.01-0.05 wt. %, 0.03-0.08 wt. %, 0.05-0.1 wt. %, 0.08-0.15 wt. %, 0.1-0.2, 0.15-0.25 wt. %, 0.2-0.3 wt. %, 0.25-0.35 wt. %, 0.3-0.4 wt. %, 0.35-0.45 wt. %, 0.4-0.5 wt. %, 0.45-0.55 wt. %, 0.5-0.6 wt. %, 0.55-0.65 wt. %, 0.6-0.7 wt. %, 0.65-0.75 wt. %, 0.7-0.8 wt. %, 0.75-0.85 wt. %, 0.8-0.9 wt. %, 0.85-0.95 wt. %, 0.9-1.0 wt. %, 0.95-1.1 wt. %, 1.0-1.2 wt. %, 1.1-1.3 wt. %, 1.2-1.4 wt. %, 1.3-1.5 wt. %, 1.4-1.6 wt. %, 1.5-1.7 wt. %, 1.6-1.8 wt. %, 1.7-1.9 wt. % and 1.8-2.0 wt. %, based on the total weight of the food contact safe composition containing an essential oil complex.

### Essential Oil

In one embodiment, the food contact safe composition containing an essential oil complex includes an essential oil. It is known in the art that many essential oils may be ingested, applied, or absorbed by a mammalian subject without causing an allergic or severe reaction. Various essential oils possess antimicrobial properties. With the inclusion of the at least one metal ion, a synergistic effect can occur between the essential oil and the at least one metal ion. The synergistic effect is exhibited by amplified antimicrobial properties of the at least one metal ion or the essential oil beyond what is expected when used alone or in combination.

Additionally, the essential oil may interact with the at least one metal ion, may prevent oxygen from interacting with the at least one metal ion, and may prevent moisture from interacting with the at least one metal ion. The essential oil may have a high propensity to form hydrogen bonds, and may have various ionic functional groups such alkenes, aromatics, ketones, carboxylates, hydroxyl groups, amine groups, sulfide groups, heterocyclic groups, or combinations thereof which interact with the metal ion through ionic and/or Van der Walls interactions. By interacting with the ionic functional groups, oxygen (O₂) and/or water may be prevented from interacting with the at least one metal ion. Thus, metal oxides from the at least one metal ions are not formed. This interaction not only stabilizes the essential oil metal ion complex, but also increases the shelf-life of the complex.

A wide variety of essential oils may be used in the food contact safe composition containing an essential oil complex. The essential oil may be a natural essential oil, a synthetic essential oil, or a derivative of an essential oil. The essential oil may be purified by means known in the art. Non-limiting examples of suitable essential oils include tea tree oil, thyme oil, eucalyptus oil, oregano oil, peppermint oil, rosemary oil, clove oil, cinnamon oil, lemongrass oil, and combinations thereof.

The wt. % of the essential oil can and will vary depending on the essential oil used in the composition, the at least one metal ion, the intended use of the composition, and the emulsifying agent. In general, the wt. % of the essential oil is an amount ranging from 1-5 wt. % based on the total weight of the food contact safe composition containing an essential oil complex. In one embodiment, the wt. % of the essential oil is present in an amount selected from the group consisting of 1-5 wt. %, 1-1.5 wt. %, 1.5-2.0 wt. %, 2-2.5 wt. %, 2.5-3 wt. %, 3-3.5 wt. %, 3.5-4 wt. %, 4-4.5 wt. %, 4.5-5 wt. %, 1-3 wt. %, 3-5 wt. % and 2-4 wt. %, based on the total weight of the food contact safe composition containing an essential oil complex.

### Emulsifying Agent

The food contact safe composition containing an essential oil complex includes an emulsifying agent which is considered non-toxic to humans. The emulsifying agent provides greater solubility of the at least one metal ion and the essential oil, aids in stabilization of the at least one metal ion, and provides an additional synergistic effect between the essential oil and the at least one metal ion. Non-limiting examples of suitable emulsifying agents include lecithin, monoglycerides, diglycerides, polysorbates, carrageenans, polyglycerols of fatty acids, sorbitol esters, beeswax, or combinations thereof. In one embodiment, the emulsifying agents are selected from the group consisting of polysorbate 80, sodium stearoyl lactylate, propylene glycol, Tween 80®, and sodium caseinate.

The wt. % of the emulsifying agent includes but is not limited to an amount ranging from 1-5 wt. % based on the total weight of the food contact safe composition containing an essential oil complex. In one embodiment, the wt. % of the emulsifying agent includes but is not limited to an amount selected from the group consisting of 1-5 wt. %, 1-1.5 wt. %, 1.5-2.0 wt. %, 2-2.5 wt. %, 2.5-3 wt. %, 3-3.5 wt. %, 3.5-4 wt. %, 4-4.5 wt. %, 4.5-5 wt. %, 1-3 wt. %, 3-5 wt. % and 2-4 wt. %, based on the total weight of the food contact safe composition containing an essential oil complex.

### Solvent

The food contact safe composition containing an essential oil complex includes a solvent. The solvent must be a solvent that is compatible with human skin and internally within the human or mammalian subject.

The solvent comprises a polar protic, a polar aprotic, a non-polar protic solvent, or a combination thereof. The solvent can and will vary depending on the components used in the food contact safe composition containing an essential oil complex. In one embodiment, the solvent is a polar protic solvent, a polar aprotic solvent, or combinations thereof. Suitable examples of polar protic solvents include, but are not limited to, water, alcohols such as methanol, ethanol, isopropanol, n-propanol, iso-butanol, n-butanol, s-butanol, t-butanol, and the like; diols such as ethylene glycol, propylene glycol; polyols such as glycerol, mannitol, sorbitol; and combinations thereof. Non-limiting examples of suitable polar aprotic solvents include but are not limited to acetonitrile N,N-dimethylacetamide (DMAC), N,N-dimethylformamide (DMF), dimethyl sulfoxide (DMSO), 1,4-dioxane, N-methyl-2-pyrrolidinone (NMP), hexamethylphosphoramide, N-methylacetamide, tetrahydrofuran (THF), 2-methyltetrahydrofuran, and combinations thereof. The polar protic solvent, the polar aprotic solvent, or a combination of the polar protic and polar aprotic solvent may be utilized with water or brine in the composition.

The solvent is present in the food contact safe composition containing an essential oil complex in an amount from 88-98 wt. %, based on the total weight of the composition containing an essential oil complex. In one embodiment, the solvent is present in the composition in an amount selected from the group consisting of 88-98 wt. %, 88-90 wt. %, 90.5-91 wt. %, 91-92 wt. %, 92.5-93 wt. %, 93.5-94 wt. %, 94.5-95 wt. %, 95.5-96 wt. %, 96.5-97 wt. %, 97.5-98 wt. %, 88-92 wt. %, 92-94 wt. %, 94-96 wt. %, and 96-98 wt. %, based on the total weight of the food contact safe composition containing an essential oil complex.

### Acceptable Excipient

The food contact safe composition containing an essential oil complex may include an acceptable excipient. The food contact safe composition containing an essential oil complex remains effective when the acceptable excipient is not included in the food contact safe composition containing an essential oil complex. With the inclusion of the acceptable excipient, a variety of products may be produced that enhance the properties of the composition containing an essential oil complex such as a synergistic effect or an elongated shelf life. Non-limiting examples of the acceptable excipient are a preservative (e.g., potassium sorbate, sorbic acid, and so forth), a stabilizer, and combinations thereof. Non-limiting examples of stabilizers include but are not limited to xanthan gum, carrageenan, guar gum, agar, locust bean gum (carob gum) cellulose gum (carboxymethyl cellulose), pectin, gum arabic, sodium alginate and or gelatin.

The wt. % of the stabilizer is in amount ranging from 1-5 wt. % based on the total weight of the food contact safe composition containing an essential oil complex. In one embodiment, the wt. % of the stabilizer is present in an amount selected from the group consisting of 1-5 wt. %, 1-1.5 wt. %, 1.5-2.0 wt. %, 2-2.5 wt. %, 2.5-3 wt. %, 3-3.5 wt. %, 3.5-4 wt. %, 4-4.5 wt. %, 4.5-5 wt. %, 1-3 wt. %, 3-5 wt. % and 2-4 wt. %, based on the total weight of the food contact safe composition containing an essential oil complex.

In general, the acceptable excipient is present in an amount ranging from 0-5 wt. % based on the total weight of the composition containing an essential oil complex. In one embodiment, the excipient is present in an amount selected from the group consisting of 0-5 wt. %, 0.5-1 wt. %, 1-1.5 wt. %, 1.5-2 wt. %, 2-2.5 wt. %, 2.5-3 wt. %, 3-3.5 wt. %, 3.5-4 wt. %, 4-4.5 wt. %, 4.5-5 wt. %, 1-3 wt. %, 3-5 wt. %, and 2-4 wt. %, based on the total weight of the composition containing an essential oil complex.

### Properties of the Food Contact Safe Composition Containing an Essential Oil Complex

The food contact safe composition containing an essential oil complex has distinctive properties.

The food contact safe composition containing an essential oil complex has a pH ranging from about 6.0 to 8.0. In one embodiment, the pH of the food contact safe composition containing an essential oil complex is in the range of 6.0 to 7.0, or 7.0 to 8.0. The range of pH is selected from the group consisting of 6.0-6.5, 6.3-6.8, 6.5-7.5, 6.8-7.8 and 7.5-8.0. By having a neutral pH, the food contact safe composition containing an essential oil complex is considered non-corrosive and non-toxic and may be used in contact with human or mammalian skin, applied directly to the skin, or may be ingested by a human or mammal.

The food contact safe composition containing an essential oil complex is heat stable and light stable. Heat stability allows the food contact safe composition containing an essential oil complex to be applied to articles above room temperature (23-28° C.) and to maintain the disinfectant/antimicrobial properties for a period of time greater than about 1 day, greater than about 7 days, greater than about 14 days, greater than about 21 days, greater than about 28 days, or even greater than about 30 days.

In one embodiment, the food contact safe compositions maintain the disinfectant/antimicrobial properties for a period of time selected from the group consisting of 12 hours to 6 months; one week to one month; one month to 6 months; three months to 6 months; one day to one month; 5-20 days; 1-10 days; 1-180 days; 15-180 days and 12-24 hours or combinations thereof.

In one embodiment, the food contact safe compositions containing an essential oil complex do not contain nanoparticles. In one embodiment, the food contact safe compositions containing an essential oil complex contains only trace amounts of nanoparticles.

The food contact safe composition containing an essential oil complex is able to maintain contact with a variety of surfaces without being corrosive or toxic. As such, the food contact safe composition containing an essential oil complex remains effective for a period greater than 1 day. In one embodiment, the food contact safe composition containing an essential oil complex would remain efficacious for a period greater than about 1 day, greater than about 7 days, greater than about 14 days, greater than about 21 days, greater than about 28 days, or even greater than about 30 days.

In one embodiment, the food contact safe compositions maintain the disinfectant/antimicrobial properties for a period of time selected from the group consisting of 12 hours to 6 months; one week to one month; one month to 6 months; three months to 6 months; one day to one month; 5-20 days; 1-10 days; 1-180 days; 15-180 days and 12-24 hours or combinations thereof.

The food contact safe composition containing an essential oil complex exhibits antimicrobial properties, antibacterial properties, antiviral properties, antifungal properties, or a combination thereof against a variety of pathogens as verified by the following tests: for bacteria and fungi: AOAC Use Dilution Method (UDM), ASTM E-2315, ISO 22196:2011; and for viruses: AATCC 100-20124, ISO18184:2019, ISO 21702:2019, Rt-PCR, liquid-liquid contact. For viruses, the Fonsum Pharma test against a Covid-19 RT-PCR evaluation was utilized. The pathogen kill rate is greater than 99% after less than a 5-minute period of time and pathogenic sterility is maintained for greater than about 1 day, where 1 day can be 24 hours.

### II. Method of Preparing a Food Contact Safe Composition Containing an Essential Oil Complex

The present invention relates to methods of preparing the food contact safe composition containing an essential oil complex, the methods comprise:
(a) combining an emulsifying agent, a solvent, and an acceptable excipient to form a mixture;
(b) combining the mixture from step (a) with an essential oil; and

The methods, as disclosed herein, may be conducted in a batch process, a semi-batch process, a semi-continuous process, or a continuous process. The methods may be conducted in the dark and/or under an inert atmosphere. Darkness and/or an inert atmosphere are not necessarily required to prepare the food contact safe composition containing an essential oil complex.

In one embodiment, a continuous process consists of combining raw materials without interruption, moving from one step to the next without waiting for other materials to be added.

In one embodiment, a batch process consists of combining raw materials together in a fixed quantity to produce a finite amount of product. All materials are added at the outset and processed until completion.

In one embodiment, a semi-batch process combines the elements of both batch and continuous processes. While some materials are continuously added, others are added in batches.

In one embodiment, a semi-continuous batch process integrates aspects of both continuous and batch process. While some production steps are continuous, others are carried out in batches. This balances efficiency and flexibility and is known in the art of production of edible oils, where extraction may be continuous but refining occurs in batches.

### Step (a): Combining the Emulsifying Agent, the Solvent, and the at Least One Acceptable Excipient to Form a Mixture

The first step in the method comprises combining the emulsifying agent, the solvent, and the one acceptable excipient to form a mixture. Emulsifying agents, the solvent, and the acceptable excipient are described in more detail above in Section (I).

In one embodiment, step (a) is conducted under an inert atmosphere. Suitable inert gases include but are not limited to helium, nitrogen, argon, and/or a combinations thereof.

In step (a), an emulsifying agent, and an acceptable excipient are added portion wise, in any sequential order, or both at the same time, to the solvent while stirring, to form a mixture. Suitable methods are known in the art for stirring mixtures, such as magnetic stirring, mechanical stirring, jet mixers, and the like.

The temperature of mixing in step (a) may range from about 50° C. to about 90° C. In one embodiment, the temperature of mixing in step (a) ranges is selected from the group consisting of about 50° C. to about 90° C., from about 55° C. to about 85° C., and from about 65° C. to about 80° C. In one embodiment, the temperature of mixing in step (a) is from about 70° C. to about 80° C.

The duration of mixing ranges from about 1 minute to about 30 minutes until a homogeneous mixture is obtained by visual determination. In one embodiment, the duration of mixing ranges is selected from the group consisting of from about 1 minute to about 30 minutes, from about 2 minutes to about 15 minutes, and from about 3 minutes to about 10 minutes until a homogeneous solution is obtained as visually determined.

Step (b): Combining the Mixture from Step (a) with an Essential Oil

The next step in the method comprises combining the mixture from step (a) with an essential oil. Essential oils are described in more detail above in Section (I).

In one embodiment, step (b) is conducted under an inert atmosphere. Suitable inert gases include but are not limited to helium, nitrogen, argon, and/or combinations thereof.

In step (b), the essential oil is added slowly, portion wise, or all at once to the mixture from step (a) while stirring. Suitable methods are known in the art for stirring mixtures, such as magnetic stirring, mechanical stirring, jet mixers, and the like.

In one embodiment, the temperature of mixing in step (b) ranges from about 50° C. to about 90° C. In one embodiment, the temperature of mixing in step (a) ranges selected from the group consisting of from about 50° C. to about 90° C., from about 55° C. to about 85° C., and from about 65° C. to about 80° C. In one embodiment, the temperature of mixing in step (b) is from about 70° C. to about 80° C.

The duration of mixing ranges from about 1 minute to about 30 minutes until a heterogeneous mixture is obtained by visual determination. In one embodiment, the duration of mixing ranges is selected from the group consisting of from about 1 minute to about 30 minutes, from about 2 minutes to about 15 minutes, and from about 3 minutes to about 10 minutes until a homogeneous solution is obtained as visually determined.

Step (c): Combining the Mixture from Step (b) with the at Least One Metal Ion

The final step in the method comprises combining the mixture from step (b) with the at least one metal ion. A list of the at least one metal ions is described in more detail above in Section (I).

In one embodiment, step (c) is conducted under an inert atmosphere. Suitable inert gases include but are not limited to helium, nitrogen, argon, and/or combinations thereof.

In step (c), the at least one metal ion is added slowly, portion wise, or all at once to the mixture from step (b) while stirring to form the composition with antimicrobial properties.

Suitable methods are known in the art for stirring mixtures, such as magnetic stirring, mechanical stirring, jet mixers, and the like.

The temperature of mixing in step (c) ranges from about 50° C. to about 90° C. In one embodiment, the temperature of mixing in step (a) ranges is selected from the group consisting of from about 50° C. to about 90° C., from about 55° C. to about 85° C., and from about 65° C. to about 80° C. In one embodiment, the temperature of mixing in step (c) is from about 70° C. to about 80° C.

The duration of mixing ranges from about 1 minute to about 30 minutes until an emulsion is obtained by visual determination. In one embodiment, the duration of mixing ranges is selected from the group consisting of from about 1 minute to 30 minutes, from about 2 minutes to 15 minutes, and from about 3 minutes to 10 minutes until an emulsion is obtained as visually determined.

After the completion of the addition of materials in step (c), the food contact safe composition containing an essential oil complex is cooled to room temperature and the food contact safe composition containing an essential oil complex is formed once cooled.

### III. Methods for Disinfecting a Surface or an Article

The present invention provides methods for disinfecting and/or maintaining the pathogenic sterility of an article. The method comprises contacting the surface of the article with the food contact safe composition containing an essential oil complex.

### Food Contact Safe Compositions Containing an Essential Oil Complex

The food contact safe composition containing an essential oil complex is described in more detail above in Section (I).

### Surfaces or Articles

In one embodiment, the article is a food container, food packaging, or a food preservative. The food contact safe composition containing an essential oil complex is applied directly to a food container or food packaging material to prevent microbial growth and extend the freshness of the food such as meat, poultry, eggs, baked goods, and cheese.

Suitable, non-limiting examples of food containers or food packaging include plastic wrap, aluminum foil, a stainless-steel container, a plastic container, a glass container, a plastic deli container, and the like.

As an antimicrobial, the food contact safe composition is directly applied to the external surface of fresh meat, fresh seafood, or processed food. Non limiting examples of meat and seafood include goat, beef, chicken, pork, turkey, duck, lobster, fish, and the like. By applying the composition, any pathogens present on the surface of a food will be eliminated.

In one embodiment, when used as a food preservative, the food contact safe composition containing an essential oil complex is sprayed on meat, fruit, baked goods, and/or a vegetables.

In one embodiment, the article is a wound covering agent such as a bandage or gauze, and the like. By applying the food contact safe composition containing an essential oil complex to a wound, the wound is adequately disinfected, allowing a significantly shorter period of time required for healing.

By applying the food contact safe composition containing an essential oil complex directly to a wound on a human or mammalian subject, the wound may be adequately disinfected, allowing a significantly shorter period of time required for healing.

In one embodiment, the article is human skin. By applying the food contact safe composition containing an essential oil complex before or after applying a cosmetic on the area of lips, eyebrows, cheek, etc., the cosmetic provides disinfecting properties to the area. Applying the Food Contact Safe Composition Containing an Essential Oil Complex

In one embodiment, the food contact safe composition containing an essential oil complex is applied in various ways. The food contact safe composition containing an essential oil complex may be rapidly sprayed, cast, or brushed in thin layers over large areas or sprayed and coated numerous times on the surface or article.

In one embodiment, the food contact safe composition is applied at a concentration of 0.1-1000 μL/cm² to 1-10 mL/cm². In one embodiment, the food contact safe composition is applied at a concentration selected from the group consisting of 0.01-1000 μL/cm², 0.05-1000 μL/cm², 0.1-1000 μL/cm², 0.5-1000 μL/cm², 1 mL/cm², 1.5 mL/cm², 0.5-1.5 mL/cm², 1-2 mL/cm², 1.5-2.5 mL/cm², 2-3 mL/cm², 2.5-3.5 mL/cm², 3-4 mL/cm², 3.5-4.5 mL/cm² 4-5 mL/cm², 4.5-5.5 mL/cm², 5-6 mL/cm², 5.5-6.5 mL/cm², 6-7 mL/cm², 6.5-7.5 mL/cm², 7-8 mL/cm², 7.5-8.5 mL/cm², 8-9 mL/cm², and 9.5-10 mL/cm², or combinations thereof.

In order to identify whether the surface or article has been fully coated, a simple touch test with a finger, a corner of a towel or cloth, etc. is utilized. If a portion of the article or the surface has not been coated, additional applications of the food contact safe composition containing an essential oil complex is reapplied to ensure full and complete coverage of the surface or article.

Various coating techniques include, but are not limited to, spray coating, dip coating, doctor-blade coating, spin coating, air knife coating, single and multilayer slide coating, gap coating, knife-over-roll coating, metering rod (Meyer bar) coating, reverse roll coating, rotary screen coating, extrusion coating, casting, using a paint brush, wiping, and/or painting.

Properties of Surfaces or Articles after Contact with the Food Contact Safe Composition Containing an Essential Oil Complex

These surfaces or articles, after being disinfected by the food contact safe composition containing an essential oil complex, kill greater than 99.9999% of the pathogens present as compared to articles that have not been treated with the complex. The surfaces or articles also possess a pleasant odor from the essential oil, maintain extended longevity of the freshness of a food product, and maintains disinfectant/antimicrobial properties of human skin contaminated with microbial growth.

In one embodiment, the surfaces or articles, after being disinfected by the food contact safe composition containing an essential oil complex, kill pathogens at a percent selected from the group consisting of more than 80%, 85%, 90%, 95%, 99% and 99.9999%.

### EXAMPLES

### Example 1: Preparation of a Food Contact Safe Antimicrobial Composition Containing an Essential Oil Complex Derived from Tween 80®, Cinnamon Oil and/or Eucalyptus Oil, Silver Nitrate, and Water

Into a round bottom flask 1.50 g of Tween 80® and 96.5 mL of distilled water are added to form a solution. This mixture is stirred using a magnetic stirrer at 75° C. for 20 minutes. Then, 2.0 g of cinnamon oil and/or eucalyptus oil are slowly added into the solution. After the addition of the oil is complete, this mixture is stirred for an additional 15 minutes at 75° C. until a homogeneous mixture is visually determined. 0.05 g of silver nitrate is slowly added to the homogeneous mixture containing the oil, polysorbate 80, and water. The mixture is stirred for 30 minutes to cool to room temperature until an emulsion is visually determined, forming the emulsifying mixture/antimicrobial composition. The emulsifying mixture/antimicrobial composition is stored in a capped glass bottle at 0° C.

An ASTM E-2315 assay is conducted under guidelines of the AOAC (Association of Official Analytical Chemists). The samples show greater than 99% reduction on exposure to Escherichia coli when exposed for just 15 seconds, thereby demonstrating instant killing activity of the composition as compared to the control. Similar tests are conducted with Staphylococcus aureus (ATCC 25923), Salmonella enterica (ATCC 51958) and Pseudomonas aeruginosa (ATCC 9027) showing the same instant kill rate of the composition as compared to the control.

### Example 2: Preparation of a Food Contact Safe Antimicrobial Composition Containing an Essential Oil Complex Derived from Tween 80®, Cinnamon Oil and/or Eucalyptus Oil, Silver Nitrate, and Water

Into a round bottom flask 1.0 g of Tween 80@ and 97.9 mL of distilled water are added to form a mixture. This mixture is stirred using a magnetic stirrer at 75° C. for 20 minutes. Then, 1.0 g of cinnamon and/or eucalyptus oil are slowly added into the solution. After the addition of the oil is complete, this mixture is stirred for an additional 15 minutes at 75° C. until a homogeneous mixture is visually determined. 0.1 g of silver nitrate is slowly added to the homogeneous mixture containing the oil, Tween 80®, and water. The mixture is stirred for 30 minutes to cool to room temperature until an emulsion is visually determined, forming the emulsifying mixture/antimicrobial composition. The emulsifying mixture/antimicrobial composition is stored in a capped glass bottle at 0° C.

An ASTM E-2315 assay is conducted under guidelines of the AOAC as described above. Similar tests are conducted using samples of the composition with Staphylococcus aureus (ATCC 25923), Salmonella enterica (ATCC 51958) and Pseudomonas aeruginosa (ATCC 9027), as described above.

### Example 3: Preparation of a Food Contact Safe Antimicrobial Composition Containing an Essential Oil Complex Derived from Polysorbate 80, Peppermint Oil and/or Eucalyptus Oil, Zinc Nitrate, and Water

Into a round bottom flask 2.0 g of polyethylene glycol and 96.4 mL of distilled water are added to form a solution. This solution is stirred using a magnetic stirrer at 75° C. for 20 minutes. Then, 1.5 g of peppermint oil and or eucalyptus oil are slowly added to the solution. After addition of the oil is complete, the mixture is stirred for an additional 15 minutes at 75° C. until a homogeneous mixture is visually determined. 0.1 g of zinc nitrate is slowly added to the homogeneous mixture containing the oil, polysorbate 80, and water. The mixture is stirred for 30 minutes to cool to room temperature until an emulsion is visually determined, forming the emulsifying mixture/antimicrobial composition. The emulsifying mixture/antimicrobial composition is stored in a capped glass bottle at 0° C.

An ASTM E-2315 assay is conducted under guidelines of the AOAC as described above. Similar tests are conducted using samples of the composition with Staphylococcus aureus (ATCC 25923), Salmonella enterica (ATCC 51958) and Pseudomonas aeruginosa (ATCC 9027), as described above.

### Example 4: Preparation of a Food Contact Safe Antiviral Composition Containing an Essential Oil Complex Derived from Polysorbate 80 or Tween 80®, Peppermint Oil And/or Eucalyptus Oil, Zinc Nitrate, and Water

Into a round bottom flask 2.0 g of polyethylene glycol and 96.4 mL of distilled water are added to form a solution. This solution is stirred using a magnetic stirrer at 75° C. for 20 minutes. Then, 1.5 g of peppermint oil and or eucalyptus oil are slowly added to the solution. After addition of the oil is complete, the mixture is stirred for an additional 15 minutes at 75° C. until a homogeneous mixture is visually determined. 0.1 g of zinc nitrate is slowly added to the homogeneous mixture containing the oil, polysorbate 80 or Tween 80®, and water. The mixture is stirred for 30 minutes to cool to room temperature until an emulsion is visually determined, forming the emulsifying mixture/antiviral composition. The emulsifying mixture/antiviral composition is stored in a capped glass bottle at 0° C. Results indicate a significant reduction in viral activity, with viruses such as MS-2 bacteriophage and feline calicivirus, demonstrating efficacy against viruses comparable to a 99% reduction in Escherichia coli or Pseudomonas aeruginosa according to ASTM E1052 standards.

Methods for Testing with Staphylococcus aureus (ATCC 6538)

Two stock solutions of E3B-2 and E3B-3 were analyzed. E3B-2 and E3B-3 are both food contact safe antimicrobial compositions containing an essential oil complex as described above, also containing polyethylene glycol, peppermint, cinnamon and/or eucalyptus oil, zinc nitrate, and water. Each solution was tested at 100% concentration and diluted with sterile water to 50% concentration. Dey-Engley (D/E) neutralizing broth was used to quench microbial activity to accurately determine bactericidal activity of each solution at selected contact times.

### Validation Methodology

### Sample Preparation:

A lyophilized pellet of bacteria (obtained from ATCC) was rehydrated with 6 mL tryptic soy broth (TSB) and incubated for 24 hours at 37° C. 1 mL of this solution was then transferred twice to TSB before initiating the experiment.

Initial microbial counts were determined before the neutralization test, at the beginning of the time-kill test, and at the end of the time-kill test. Using buffered water diluent, the microorganisms were recovered and enumerated on 3M Plate Count Petrifilm™ and incubated at 35±2° C. for 48 hours.

### Neutralization

Prior to the trial, the effective neutralization of the solution was confirmed. Four test tubes, simulating each testing condition were combined with 0.1 mL of 10⁸ CFU/mL inoculum suspension. Immediately, 1 mL from each solution was added to Butterfield's Buffered Phosphate Diluent and 1 mL was added to D/E neutralizing broth. Samples were plated in duplicate on 3M Petrifilm™

### Neutralization Results

At least 95% of the microorganisms introduced to each solution were recovered. The D/E neutralization broth was confirmed to sufficiently quench the anti-microbial activity under all testing conditions (Table 1). This allowed for D/E broth to be used to stop microbial growth immediately, at the time intervals chosen.

TABLE 1
Neutralization using D/E Broth and solutions of
E3B-2 or E3B-3 with Staphylococcus aureus

| | | |
|---|---|---|
| Replicates | D/E Neutralizing Broth | Buffered Water |

100% E3B-3

| | | | | |
|---|---|---|---|---|
| 1 | 77000000 CFU/g | 7.89* | 4300000 CFU/g | 6.63* |
| 2 | 74000000 CFU/g | 7.87* | 3300000 CFU/g | 6.52* |
| Average | 75500000 CFU/g | 7.88* | 3800000 CFU/g | 6.58* |

50% E3B-3

| | | | | |
|---|---|---|---|---|
| 1 | 29100000 CFU/g | 7.46* | 3160000 CFU/g | 6.50* |
| 2 | 26700000 CFU/g | 7.43* | 4900000 CFU/g | 6.69* |
| Average | 27900000 CFU/g | 7.45* | 4030000 CFU/g | 6.61* |

100% E3B-2

| | | | | |
|---|---|---|---|---|
| 1 | 45000000 CFU/g | 7.65* | 480000 CFU/g | 5.68* |
| 2 | 40000000 CFU/g | 7.60* | 470000 CFU/g | 5.67* |
| Average | 42500000 CFU/g | 7.63* | 475000 CFU/g | 5.68* |

50% E3B-2

| | | | | |
|---|---|---|---|---|
| 1 | 59000000 CFU/g | 7.77* | 67000 CFU/g | 4.83* |
| 2 | 40000000 CFU/g | 7.60* | 68000 CFU/g | 4.83* |
| Average | 49500000 CFU/g | 7.69* | 67500 CFU/g | 4.83* |

Results are shown in CFU/g and converted into *Log₁₀ count

### Time Kill Test

Each stock solution and subsequent concentration was challenged with 0.1 mL of 10⁸ CFU/mL inoculum suspension. The tested contact times were one minute, five minutes, and ten minutes. Anti-microbial activity was quenched by adding 1 mL of each suspension to 9 mL of D/E broth. The number of surviving microorganisms were compared to the initial microorganism counts to determine the log reduction of the two stock solutions at two concentrations of E3B-2 and E3B-3.

### E3B-2 Results

After one minute of contact time the E3B-2 100% mixture achieved a 3.73 log reduction, and the 50% mixture achieved a 3.75 log reduction in microorganisms counts (Tables 1A and 1B). After five minutes the reduction increased to 4.78 and 4.76 respectively. After ten minutes of contact time a 5.84 and 5.61 log reduction were achieved.

TABLE 1A
Stock solution E3B-2 at 100% Concentration
with Staphylococcus aureus

| | | | | | Percent |
|---|---|---|---|---|---|
| | | | | Standard | reduction in |
| Time | Surviving | Log₁₀ | Log | Error of | number of |
| Interval | Colonies | Counts | Reduction | the Mean | colonies |
| 1 min. | 61000 | 4.79 | −3.75 | | |
| Average | 68000 | 4.83 | −3.70 | | |
| Variance | 64500 | 4.81 | −3.73 | 0.02 | 99.98 |
| | 1.225E04 | 0.0006 | 0.0006 | | |
| 5 min. | 5800 | 3.76 | −4.77 | | |
| Average | 5600 | 3.75 | −4.79 | | |
| Variance | 5700 | 3.76 | −4.78 | 0.01 | 99.9979 |
| | 10000 | 5.81E−05 | 5.81E−05 | | |
| 10 min. | 600 | 2.78 | −5.76 | | |
| Average | 400 | 2.60 | −5.94 | | |
| Variance | 500 | 2.70 | −5.84 | | |
| | 10000 | 0.0078 | 0.0078 | 0.06 | 99.999 |

TABLE 1B
Stock solution E3B-2 at 50% Concentration
with Staphylococcus aureus

| | | | | | Percent |
|---|---|---|---|---|---|
| | | | | Standard | reduction in |
| Time | Surviving | Log₁₀ | Log | Error of | number of |
| Interval | Colonies | Counts | Reduction | the Mean | colonies |
| 1 min. | 64000 | 4.81 | −3.73 | | |
| Average | 59000 | 4.77 | −3.77 | | |
| Variance | 61500 | 4.79 | −3.75 | 0.02 | 99.98 |
| | 6.25E6 | 0.0003 | 0.0003 | | |
| 5 min. | 5800 | 3.76 | −4.77 | | |
| Average | 6300 | 3.80 | −4.74 | | |
| Variance | 6050 | 3.78 | −4.76 | 0.02 | ≈99.997 |
| | 62500 | 0.0003 | 0.0003 | | |
| 10 min. | 1200 | 3.08 | −5.46 | | |
| Average | 500 | 2.70 | −5.84 | | |
| Variance | 850 | 2.93 | −5.61 | 0.14 | 99.999 |
| | 122500 | 0.0361 | 0.0361 | | |

### E3B-2 Discussion

The standard error of the mean for all contact times and both concentrations was low. The highest variation recorded was 0.14 between the ten-minute contact time samples of the 50% concentration solution. At the end of the experiment the percent reduction in surviving microorganisms was 100% when rounding to the hundredth place for both concentrations of the solution.

### E3B-3 Results

After one minute of contact time the E31B-3 100% concentration achieved a 3.72 log reduction, and the 5000 concentration achieved a 3.36 log reduction (Tables 1C and 1D). After five minutes of contact time the log reduction increased to 5.01 for the 100% concentration and decreased to 3.36 for the 50% concentration. After ten minutes of contact time the 100% concentration achieved a 6.54 log reduction in surviving microorganisms while the 50% concentration achieved a 4.95 log reduction.

TABLE 1C
Stock solution E3B-3 at 100% Concentration
with Staphylococcus aureus

| | | | | | Percent |
|---|---|---|---|---|---|
| | | | | Standard | reduction in |
| Time | Surviving | Log₁₀ | Log | Error of | number of |
| Interval | Colonies | Counts | Reduction | the Mean | colonies |
| 1 min. | 73000 | 4.86 | −3.67 | | |
| Average | 57000 | 4.76 | −3.78 | | |
| Variance | 65000 | 4.81 | −3.72 | 0.04 | 99.98 |
| | 6.4E7 | 0.003 | 0.003 | | |
| 5 min. | 3100 | 3.49 | −5.05 | | |
| Average | 3600 | 3.56 | −4.98 | | |
| Variance | 3350 | 3.53 | −5.01 | 0.03 | 99.9989 |
| | 62500 | 0.001 | 0.001 | | |
| 10 min. | 100 | 2.00 | −6.54 | | |
| Average | 100 | 2.00 | −6.54 | | |
| Variance | 100 | 2.00 | −6.54 | 0.01 | 99.999999 |
| | 0 | 0.000 | 0.000 | | |

TABLE 1D
Stock solution E3B-3 at 50% Concentration
with Staphylococcus aureus

| | | | | | Percent |
|---|---|---|---|---|---|
| | | | | Standard | reduction in |
| Time | Surviving | Log₁₀ | Log | Error of | number of |
| Interval | Colonies | Counts | Reduction | the Mean | colonies |
| 1 min. | 156000 | 5.19 | −3.34 | | |
| Average | 147000 | 5.17 | −3.37 | | |
| Variance | 151500 | 5.18 | −3.36 | 0.02 | 99.96 |
| | 20250000 | 0.0002 | 0.0002 | | |
| 5 min. | 231000 | 5.36 | −3.17 | | |
| Average | 171000 | 5.23 | −3.30 | | |
| Variance | 201000 | 5.30 | −3.23 | 0.05 | 99.94 |
| | 900000000 | 0.0043 | 0.0043 | | |
| 10 min. | 3700 | 3.57 | −4.97 | | |
| Average | 4100 | 3.61 | −4.92 | | |
| Variance | 3900 | 3.59 | −4.95 | 0.02 | 99.9984 |
| | 40000 | 0.0005 | 0.0005 | | |

### E3B-3 Discussion

The variance in this trial was lower than the previous trial with the largest standard error of the mean being 0.05 between the five-minute contact time samples at 50% concentration. At the end of the experiment both concentrations achieved a 100% reduction in surviving microorganisms when rounding to the hundredth place.

### Conclusion

After ten minutes of contact time both concentrations of E3B-2 achieved a 5-log reduction against S. aureus based on the data presented.

After at least five minutes of contact time the 100% concentration of E3B-3 achieved a 5.01 log reduction against S. aureus. The 50% concentration of this solution achieved a 4.95 log reduction in counts after ten minutes of contact time.

Methods for Testing with Salmonella enterica (ATCC 51958)

Two stock solutions of E3B-2 and E3B-3 were analyzed as described in [00109] above.

### Sample Preparation

A lyophilized pellet from ATCC was rehydrated as described in [00110] above.

Initial microbial counts were determined before the neutralization test as described in [00111] above.

### Neutralization

Prior to the trial, the effective neutralization of the stock solution was confirmed as described in [00112] above.

### Neutralization Results

At least 95% of the microorganisms introduced to each solution were recovered. The D/E neutralization broth was confirmed to sufficiently quench the anti-microbial activity of all four testing conditions (Table 2). This allowed for D/E broth to be used to stop microbial kill immediately, at the time intervals chosen.

TABLE 2
Neutralization using D/E Broth and solutions
of E3B-2 or E3B-3 with Salmonella Enterica

| | | |
|---|---|---|
| Replicates | D/E Neutralizing Broth | Buffered Water |

E3B-2 (100%)

| | | | | |
|---|---|---|---|---|
| 1 | 7900000 CFU/g | 6.90* | 8900000 CFU/g | 6.95* |
| 2 | 8400000 CFU/g | 6.92* | 5300000 CFU/g | 6.72* |
| Average | 8150000 CFU/g | 6.91* | 7100000 CFU/g | 6.85* |

E3B-2 (50%)

| | | | | |
|---|---|---|---|---|
| 1 | 8000000 CFU/g | 6.90* | 8800000 CFU/g | 6.94* |
| 2 | 8400000 CFU/g | 6.92* | 7600000 CFU/g | 6.88* |
| Average | 8200000 CFU/g | 6.91* | 8200000 CFU/g | 6.91* |

E3B-3 (100%)

| | | | | |
|---|---|---|---|---|
| 1 | 8500000 CFU/g | 6.93* | 10300000 CFU/g | 7.01* |
| 2 | 7600000 CFU/g | 6.88* | 9200000 CFU/g | 6.96* |
| Average | 8050000 CFU/g | 6.91* | 9750000 CFU/g | 6.99* |

E3B-3 (50%)

| | | | | |
|---|---|---|---|---|
| 1 | 9300000 CFU/g | 6.97* | 8500000 CFU/g | 6.93* |
| 2 | 9100000 CFU/g | 6.96* | 9200000 CFU/g | 6.96* |
| Average | 9200000 CFU/g | 6.96* | 8850000 CFU/g | 6.95* |

Results are shown in CFU/g and converted into *Log10 count.

### Time Kill Test

Each stock solution and subsequent concentration was challenged with 0.1 mL of 10⁸ CFU/mL inoculum suspension. The tested contact times were one minute, five minutes, and ten minutes. Anti-microbial activity was quenched by adding 1 mL of each suspension to 9 mL of D/E neutralization broth. The number of surviving microorganisms were compared to the initial microorganism counts to determine the log reduction of the two stock solutions at two concentrations of E3B-2 and E3B-3.

### E3B-2 Results

After one minute of contact time the E3B-2 10000 concentration achieved a 3.81 log reduction, and the 5000 concentration achieved a 3.88 log reduction in microorganisms counts (Tables 2A and 2B). After five minutes the reduction increased to 3.95 for the 100% concentration and decreased to 3.86 for the 50% concentration. After ten minutes of contact time a 4.73 and 4.71 log reduction were achieved.

TABLE 2A
Stock solution E3B-2 at 100% Concentration with Salmonella Enterica

| | | | | | Percent |
|---|---|---|---|---|---|
| | | | | Standard | Reduction in |
| Time | Surviving | Log₁₀ | Log | Error of | number of |
| Interval | Colonies | Counts | Reduction | the Mean | colonies |
| 1 min. | 70000 | 4.85 | −3.86 | | |
| Average | 89000 | 4.95 | −3.76 | | |
| Variance | 79500 | 4.90 | −3.81 | 0.037 | 99.98 |
| | 9.025E7 | 0.0027 | 0.0027 | | |
| 5 min. | 57000 | 4.76 | −3.95 | | |
| Average | 56000 | 4.75 | −3.96 | | |
| Variance | 56500 | 4.75 | −3.95 | 0.003 | 99.99 |
| | 250000 | 1.47718E−05 | 1.47718E−05 | | |
| 10 min. | 9200 | 3.96 | −4.74 | | |
| Average | 9600 | 3.98 | −4.72 | | |
| Variance | 9400 | 3.97 | −4.73 | | |
| | 40000 | 8.5E−05 | 8.54E−05 | 0.007 | 99.9977 |

TABLE 2B
Stock solution E3B-2 at 50% Concentration
with Salmonella Enterica

| | | | | | Percent |
|---|---|---|---|---|---|
| | | | | Standard | reduction in |
| Time | Surviving | Log₁₀ | Log | Error of | number of |
| Interval | Colonies | Counts | Reduction | the Mean | colonies |
| 1 min. | 71000 | 4.85 | −3.86 | | |
| Average | 63000 | 4.80 | −3.91 | | |
| Variance | 67000 | 4.83 | −3.88 | 0.02 | 99.99 |
| | 1.6E7 | 0.0007 | 0.0007 | | |
| 5 min. | 75000 | 4.88 | −3.83 | | |
| Average | 65000 | 4.81 | −3.89 | | |
| Variance | 70000 | 4.85 | −3.86 | 0.02 | 99.99 |
| | 2.5E7 | 0.0010 | 0.0010 | | |
| 10 min. | 10600 | 4.03 | −4.68 | | |
| Average | 9200 | 3.96 | −4.74 | | |
| Variance | 9900 | 4.00 | −4.71 | 0.02 | ≈99.9976 |
| | 490000 | 0.0009 | 0.0009 | | |

### E3B-2 Discussion

The standard error of the mean for all contact times and both concentrations was low. The highest variation recorded was 0.037 between the one-minute contact time samples of the 1000% concentration solution. At the end of the experiment the percent reduction in surviving microorganisms was 100% when rounding to the hundredth place for both concentrations of the solution.

### E3B-3 Results

After one minute of contact time the E3B-3 100% concentration achieved a 3.88 log reduction, and the 50% concentration achieved a 3.32 log reduction (Tables 2C and 2D3). After five minutes of contact time the log reduction increased to 4.07 for the l100% concentration and decreased to 3.19 for the 5000 concentration. After ten minutes of contact time the 100% concentration achieved a 6.71 log reduction in surviving microorganisms while the 50% concentration achieved a 4.88 log reduction.

TABLE 2C
Stock solution E3B-3 at 100% Concentration
with Salmonella Enterica

| | | | | | Percent |
|---|---|---|---|---|---|
| | | | | Standard | reduction in |
| Time | Surviving | Log₁₀ | Log | Error of | number of |
| Interval | Colonies | Counts | Reduction | the Mean | colonies |
| 1 min. | 61000 | 4.79 | −3.92 | | |
| Average | 72000 | 4.86 | −3.85 | | |
| Variance | 66500 | 4.82 | −3.88 | 0.03 | 99.99 |
| | 30250000 | 0.0013 | 0.0013 | | |
| 5 min. | 46000 | 4.66 | −4.04 | | |
| Average | 41000 | 4.61 | −4.09 | | |
| Variance | 43500 | 4.64 | −4.07 | 0.03 | 99.99 |
| | 6250000 | 0.0006 | 0.0006 | | |
| 10 min. | 1780 | 3.25 | −5.46 | | |
| Average | 2240 | 3.35 | −5.36 | | |
| Variance | 100 | 2.00 | −6.71 | 0.04 | 99.9999 |
| | 52900 | 0.002 | 0.002 | | |

TABLE 2D
Stock solution E3B-3 at 50% Concentration
with Salmonella Enterica

| | | | | | Percent |
|---|---|---|---|---|---|
| | | | | Standard | reduction in |
| Time | Surviving | Log₁₀ | Log | Error of | number of |
| Interval | Colonies | Counts | Reduction | the Mean | colonies |
| 1 min. | 300000 | 5.48 | −3.23 | | |
| Average | 193000 | 5.29 | −3.42 | 0.07 | 99.95 |
| Variance | 246500 | 5.39 | −3.32 | | |
| | 2.86225E9 | 0.01 | 0.01 | | |
| 5 min. | 187000 | 5.27 | −3.43 | | |
| Average | 470000 | 5.67 | −3.03 | | |
| Variance | 328500 | 5.52 | −3.19 | 0.14 | 99.94 |
| | 2.002225E10 | 0.04 | 0.04 | 0.01 | 99.9984 |
| 10 min. | 6400 | 3.81 | −4.90 | | |
| Average | 6900 | 3.84 | −4.87 | | |
| Variance | 6650 | 3.82 | −4.88 | | |
| | 62500 | 0.0003 | 0.0003 | | |

### Discussion

The variance in this trial was higher than the previous trial with the largest standard error of the mean being 0.14 between the five-minute contact time samples at 50% concentration. At the end of the experiment both concentrations achieved a 100% reduction in surviving microorganisms when rounding to the hundredth place.

### Conclusion

After ten minutes of contact time E3B-3 at 100% concentration achieved at least a 5-log reduction against S. enterica. This was the only test solution that achieved greater than a 5-log reduction. The 50% concentration of E3B-3 achieved a 4.88 log reduction. E3B-2 at 100% concentration achieved a 4.73 log reduction and 50% achieved a 4.71 log reduction.

In order to achieve the desired log reduction both concentrations of E3B-2 and 50% concentration of E3B-3 would need a longer contact time with the inoculum. The data suggests that a 5-log reduction is achievable and 10 minutes of contact time for both solution concentrations is less than half a log away from the goal reduction.

What is claimed is:

1. A food contact safe composition containing an essential oil complex for killing pathogens comprising: from about 0.01 weight percent (wt. %) to 0.1 wt. % of at least one metal ion; from about 1.0 wt. % to 5.0 wt. % of an essential oil; from about 1.0 wt. % to 5.0 wt. % of an emulsifying agent; and from about 89.9 wt. % to 98.9 wt. % of a solvent.

2. The food contact safe composition of claim 1, wherein the at least one metalion is a food safe metal ion.

3. The food contact safe composition of claim 1, wherein the at least one metalion is a water-soluble metal salt.

4. The food contact safe composition of claim 3, wherein the at least one metal ion comprises a dissociated silver salt, a dissociated copper salt, a dissociated zinc salt, a dissociated gold salt, a dissociated cobalt salt, a dissociated zirconium salt, a dissociated molybdenum salt, a dissociated palladium salt, or a combination of dissociated salts thereof.

5. The food contact safe composition of claim 1, wherein the essential oil is selected from a group consisting of tea tree oil, thyme oil, eucalyptus oil, oregano oil, peppermint oil, rosemary oil, clove oil, cinnamon oil, lemongrass oil, and a combination thereof.

6. The food contact safe composition of claim 1, wherein the emulsifying agent comprises lecithin, a monoglyceride, a diglyceride, a polysorbate, a carrageenan, a polyglycerol of a fatty acid, a sorbitol ester, a beeswax, or combinations thereof.

7. The food contact safe composition of claim 6, wherein the emulsifying agent comprises polysorbate 80, sodium stearoyl lactylate, propylene glycol, Tween 80®, or sodium caseinate and combinations thereof.

8. The food contact safe composition of claim 1, wherein the solvent comprises a polar protic, a polar aprotic, a non-polar protic solvent, or a combination thereof.

9. The food contact safe composition of claim 8, wherein the solvent comprises water.

10. The food contact safe composition of claim 1, further comprising an acceptable excipient selected from a group consisting of a preservative, a stabilizer, and combinations thereof.

11. The food contact safe composition of claim 1, wherein the food contact safe composition has a pH range selected from the group consisting of about 6.0 to 8.0, about 6.0 to 7.0, and about 7.0 to 8.0.

12. The food contact safe composition of claim 1, wherein the food contact safe composition is non-toxic and non-corrosive.

13. The food contact safe composition of claim 1, wherein the essential oil prevents the at least one metal salt from oxidizing.

14. The food contact safe composition of claim 1, wherein the food contact safe composition is non-toxic and non-corrosive.

15. The food contact safe composition of claim 1, wherein the food contact safe composition is light and heat stable.

16. The food contact safe composition of claim 1, wherein the essential oil prevents oxidation of the at least one metal ion.

17. The food contact safe composition of claim 1, wherein the food contact safe composition maintains contact on a surface.

18. The food contact safe composition of claim 1, wherein the food contact safe composition exhibits antimicrobial properties, antibacterial properties, antifungal properties, antiviral properties, or a combination thereof against a variety of pathogens.

19. The food contact safe composition of claim 20, wherein the food contact safe composition exhibits a greater than 99% kill rate on a surface in less than 3 minutes.

20. The food contact safe composition of claim 1, wherein the food contact safe composition maintains efficacy for greater than 1 day.

21. A method for preparing a food contact safe composition containing an essential oil complex for killing pathogens comprising:
(a) contacting an emulsifying agent, a solvent, and one acceptable excipient forming a mixture;
(b) contacting the mixture from step (a) with an essential oil; and
(c) contacting the mixture from step (b) with at least one metal ion;
wherein the food contact safe composition containing an essential oil complex does not contain nanoparticles.

22. The method of claim 21, wherein the at least the one metal ion is a food safe metal ion.

23. The method of claim 21, wherein the at least the one metal ion comprises at least one water soluble metal salt.

24. The method of claim 21, wherein the at least one metal ion comprises a silver salt, a copper salt, a zinc salt, a gold salt, a cobalt salt, a nickel salt, a zirconium salt, a molybdenum salt, a palladium salt, or a combination thereof.

25. The method of claim 21, wherein the essential oil is selected from a group consisting of tea tree oil, thyme oil, eucalyptus oil, oregano oil, peppermint oil, rosemary oil, clove oil, cinnamon oil, lemongrass oil, and a combination thereof.

26. The method of claim 21, wherein the emulsifying agent comprises lecithin, a monoglyceride, a diglyceride, a polysorbate, a carrageenan, a polyglycerol of a fatty acid, a sorbitol ester, a beeswax, or combinations thereof.

27. The method of claim 21, wherein the emulsifying agent comprises polysorbate 80, sodium stearoyl lactylate, propylene glycol, Tween 80®, and sodium caseinate.

28. The method of claim 21, wherein the solvent comprises a polar protic, a polar aprotic, a non-polar protic solvent, or a combination thereof.

29. The method of claim 28, wherein the solvent comprises water.

30. The method of claim 21, further comprising an acceptable excipient selected from the group consisting of a preservative, a stabilizer, and combinations thereof.

31. The method of claim 21, wherein the food contact safe composition has a pH range selected from the group consisting of about 6.0 to 8.0, about 6 to 7, and about 7 to 8.

32. The method of claim 21, wherein the method is conducted from 50° C. to about 90° C.

33. The method of claim 21, wherein the method is conducted at 70° C.

34. The method of claim 21, wherein the method is conducted in a batch process, a semi-batch process, a semi-continuous process, or a continuous process.

35. The method of claim 21, wherein the food contact safe composition is non-toxic and non-corrosive.

36. The method of claim 21, wherein the food contact safe composition is light and heat stable.

37. The method of claim 21, wherein the essential oil prevents the at least one metal ion from oxidizing.

38. The method of claim 21, wherein the food contact safe composition maintains contact on a variety of surfaces.

39. The method of claim 21, wherein the food contact safe composition exhibits antimicrobial properties, antibacterial properties, antifungal properties, antiviral properties, or a combination thereof against a variety of pathogens.

40. The method of claim 21, wherein the food contact safe composition achieves a greater than a 99% kill rate on a surface in less than 3 minutes.

41. A method of disinfecting and/or maintaining a pathogenic sterility of an article comprising contacting the article with the food contact safe composition containing an essential oil complex of claim 1.

42. The method of claim 41, wherein the article comprises a food product, a cosmetic, or a pharmaceutical.

43. The method of claim 41, wherein the method kills greater than 99% of pathogens on the surface of the article.

44. The method of claim 41, wherein the disinfected article maintains a reduction of pathogens and/or efficacy for greater than 1 day.
